(19) United States
(12) Patent Application Publication
(10) Pub. No.: US 2013/0115318 A1
(43) Pub. Date: May 9, 2013

(54) COSMETIC COMPOSITION CONTAINING A CYPRESS ESSENTIAL OIL COMPLEX AS AN ACTIVE INGREDIENT FOR ENHANCING MEMORY AND IMPROVING COGNITIVE DYSFUNCTION

(71) Applicant: Su-Kang Han, Seoul (KR)

(72) Inventor: Su-Kang Han, Seoul (KR)

(73) Assignee: SKINBIO CO.,LTD., Hwaseong, Gyeonggi-Do (KR)

(21) Appl. No.: 13/698,594

(22) PCT Filed: May 16, 2011
(86) PCT No.: PCT/KR2011/003597

§ 371 Date: Jan. 14, 2013

(30) Foreign Application Priority Data
May 19, 2010 (KR) 10-2010-0046784

(51) Int. Cl. A61K 9/00

(52) U.S. Cl. CPC A61K 9/0012
USPC 424/745

(57) ABSTRACT

The present invention relates to a cosmetic composition containing cypress essential oil as a first essential oil and one or more second essential oils selected from the group consisting of pine needle essential oil, lavender essential oil, rosemary essential oil, red thyme essential oil, and bergamot essential oil. The composition has the effects of enhancing memory and reducing cognitive dysfunction by means of the fragrance thereof instead of via oral administration.

### CROSS-REFERENCES TO RELATED APPLICATIONS

This application is a U.S. national phase application, pursuant to 35 U.S.C. §371, of PCT/KR2011/003597, filed May 16, 2011, designating the United States, which claims priority to Korean Application No. 10-2010-0046784, filed May 19, 2010. The entire contents of the aforementioned patent applications are incorporated herein by this reference.

### TECHNICAL FIELD

The present invention relates to a cosmetic composition comprising a cypress essential oil as an active ingredient for enhancing memory and improving cognitive functions.

### BACKGROUND ART

Dementia is a complex clinical syndrome characterized by a serious loss of global cognitive ability, intellectual capability, and emotional and behavior control. Since dementia is caused by diseases that affect the brain areas responsible for memory, attention, language, and orientation in space and time, it is almost impossible for the patients to do everyday and social life tasks. On the whole, dementia results from the impairment of one or more cognitive functions, but is different from a mere problem of memory.

There are various etiologies of dementia including neurodegenerative diseases such as Alzheimer's disease and Parkinson's disease, vascular diseases such as cerebral hemorrhage, metabolic diseases such as hepatic encephalopathy and Wilson's disease, infectious diseases such as neurosyphilis and AIDS, drug addiction such as alcohol addiction, and trauma such as traumatic brain injury. So long as it brings about structural or functional abnormality in the central nervous system, a disease, whether in any form, may be apt to cause dementia. Alzheimer's disease is the most common form of dementia, accounting for 50˜60% of the cases, followed by vascular dementia.

Of the elderly population over 65 years of age in South Korea, 0.46 million are affected by dementia as of 2010, with the prediction of a sharp increase in numbers to 0.7 million by 2020, and 2 million by 2040. Due to requirement for long-term care and the characteristic symptoms thereof, patients with dementia suffer from far greater inconvenience in everyday life than do patients with other severe diseases, with great physical, psychological and economic burdens imposed thereon. In consideration of the increase in geriatric population and the prevalence rate of dementia, there is a need for a social and national solution to problems with dementia.

Memory impairment is the initial and most common symptom that Alzheimer' disease patients suffer from. In the early stages of Alzheimer's disease, the patients suffer from the impairment of recent memory, that is, have difficulty in remembering recent events, such as dialogues, details, etc. that have recently took place. This is attributed to damage of the hippocampus which results in the loss of ability to form new memories, although older memories are safe. However, the remote (long-term) memory is increasingly lost with the gradual progress of the disease because the cerebral cortex responsible for the storage of long-term memory is impaired.

Symptoms of Alzheimer's disease are closely associated with cholinergic synaptic dysfunction as well as the cytotoxic deposition of β-amyloid. Cholinergic dysfunction is known to contribute to memory impairment and cognitive dysfunction. In cooperation with the temporal lobe, hippocampus, and amygdala, the cholinergic neurons of the basal nucleus of Meynert in the basal forebrain is responsible for memory and cognitive ability. In the brains of Alzheimer's disease patients, the concentration of cholinergic neurons is reported to decrease to 78% in temporal lobe, 60% in hippocampus, and 67% in the basal nucleus of Meynert, compared to normal persons. Given a cytotoxic injury, brain cells suffer from impairment in the transmission of information, that is, the metabolism of neurotransmitters, culminating in memory and cognitive impairment. Much research has already reported that levels of acetylcholine and an enzyme associated with the synthesis thereof (choline acetyltransferase) are selectively reduced in the brain of Alzheimer's disease patients. Further, the brain of Alzheimer's disease patients not only decreases in the level of both nicotinic acetylcholine receptor and muscarinic acetylcholine receptor, but also shows poor functions of resorbing choline and secreting acetylcholine, compared to that of normal persons.

Various drugs have been developed to reverse the impairment of cholinergic neurons. The most effective and popular among them are acetylcholinesterase inhibitors (AchEI). As such, donepezil, rivastigmine, and galanthamine are currently marketed with the permission of the FDA. These drugs delay the breakdown of acetylcholine released into synaptic clefts to increase synaptic density, thus exerting a therapeutic effect. The acetylcholinesterase inhibitors are used only in the palliative treatment of mild to moderate Alzheimer's disease because the patients are improved cognitive ability and behavior of everyday life for an initial duration of administration with the drugs, but return to the pre-administration states after administration for 9 months to one year. Thus, these drugs cannot block the progression of the disease. Only when the drugs are applied in the early stage of Alzheimer's disease can their therapeutic effects be significant. Almost no or little therapeutic effects are exhibited in severe dementia. Their common side effects include nausea, diarrhea, anorexia, dizziness, muscle cramps, and sleep disturbance, and in the worst case, sinus bradycardia with swooning is caused. Memantine (NMDA receptor blocker) preventive of neurological impairment, a ginkgo (ginko biloba) extract protective of neurons, and huperzine A (reversible AchEI), an herbal extract, are also known as therapeutic agents for Alzheimer's disease. In addition, extensive research has been directed toward the development of therapeutic agents interruptive of the pathogenic mechanism of Alzheimer's disease, such as factors implicated in the formation of tau protein, the prevention of the formation and deposition of beta-amyloid protein, antioxidation, anti-inflammatory activity, etc. The South Korean market of therapeutics for Alzheimer's disease is monopolized in practice. Over 98% of the Alzheimer's disease therapeutics currently marketed in South Korea are products of foreign companies, with one company having 80% of the Korean market.

Various extracts from natural herbs are suggested as therapeutics for Alzheimer's disease. For example, Korean Patent Laid-Open Publication No. 10-2009-0082946 discloses a mixture of a green tea extract and theanine at a certain ratio as an active ingredient of a composition for enhancing memory and cognitive ability. Korean Patent No. 10-0360674 describes a composition for the prevention and treatment of dementia, comprising an extract from Pleuropterus multiflorus TURCZ as an active ingredient. Korean Patent No. 10-0822887 provides a composition comprising a mixture of a Chelidonium majus var. asiaticum extract, 8-hydroxydihydrochelerythrine, 8-hydroxydihydrosanguinarine, and berberine for the treatment or prevention of dementia. In addition, Korean Patent No. 10-0804480 concerns a composition for enhancing memory, comprising a mixture of extracts from Polygala tatarinowi REGEL, Acorus gramineus Solander, ginseng, Angelica gigas Nakai, ginkgo leaves, Schizandra chinensis BAALL, and Cnidium officinale MAKINO. Korean Patent Laid-Open Publication No. 10-2005-0092292 describes an herbal composition for enhancing memory, comprising extracts from Atractylodes macrocephala Koidzumi, Poria cocos Wolf, and Zizyphus jujuba Miller. An herbal composition for the enhancement of memory comprising a Rehmanniae radix preparata extract is described in Korean Patent No. 10-0500029. Conventional herbal compositions for enhancing memory or treating dementia are, for the most part, formulated into oral dosage forms, which require oral administration at regular intervals in daily life, causing inconvenience to the patients.

### DISCLOSURE

### Technical Problem

It is an object of the present invention to provide a cosmetic composition for the enhancement of memory and improving cognitive dysfunction by means of fragrance, instead of via oral administration.

### Technical Solution

In accordance with an aspect thereof, the present invention provides a cosmetic composition for enhancing memory and improving cognitive dysfunction, comprising a first essential oil component composed of a cypress essential oil, and a second essential oil component selected from the group consisting of a pine needle essential oil, a lavender essential oil, a rosemary essential oil, a red thyme essential oil, a bergamot essential oil, and a combination thereof.

In one embodiment of the present invention, the first essential oil component and the second essential oil component are used together in an amount of from 0.01% by weight to 100% by weight based on the total weight of the composition.

In another embodiment of the present invention, the second essential oil component is used in an amount of from 0.01 part by weight to 100 parts by weight based on 100 parts by weight of the first essential oil component.

In a further embodiment of the present invention, the cosmetic composition may further comprise an additive selected from the group consisting of a carrier, an excipient, a diluent, a filler, a thickener, a wetting agent, a lubricant, a disintegrant, a surfactant, and a combination thereof.

In a still further embodiment of the present invention, the cosmetic composition is preferably in a formulation form selected from the group consisting of shampoo, perfume, lotion, cream, skin lotion, wash, mousse, spray, hair wax, deodorant, aromatic, and hair gel.

### Advantageous Effects

Having a therapeutic effect on scopolamine-induced memory impairment and cognitive dysfunction, the cosmetic composition comprising an aromatic cypress essential oil complex in accordance with the present invention is useful in the enhancement of memory and the prevention and treatment of cognitive dysfunction. The cypress essential oil complex was found to have better therapeutic effects than did individual essential oils, and far better than artificial aromatics, as measured by various cognitive function tests.

### BRIEF DESCRIPTION OF DRAWINGS

FIGS. 1 and 2 are graphs showing percentage of spontaneous alteration and the total number of arm entries, respectively, as results of Experimental Example 1.

FIGS. 3 and 4 are graphs showing mean escape latency according to test groups and training days, respectively, as results of Experimental Example 2.

FIGS. 5 and 6 are graphs showing step-through latency time during a retention trial on day 2 and during an acquisition trial on day 1, respectively, as results of Experimental Example 3.

FIGS. 7 and 8 are graphs showing time of freezing behavior in contextual fear conditioning and in cued fear conditioning, respectively, as results of Experimental Example 4.

### BEST MODE

In accordance with an aspect thereof, the present invention addresses an aromatic composition comprising a first essential oil component composed of cypress essential oil, and a second essential oil component selected from the group consisting of a pine needle essential oil, a lavender essential oil, a rosemary essential oil, a red thyme essential oil, and a combination thereof. As used herein, the term “essential oil” refers to a natural oil extracted from plants.

With regard to the mechanism of the cosmetic composition of the present invention for enhancing memory and preventing and treating cognitive dysfunction, it is characterized by increasing expressions of neurotropic factors, choline acetyltransferase, and acetylcholine receptors, and by inhibiting acetylcholinesterase. Examples of the cognitive dysfunction include dementia of Alzheimer type, vascular dementia, and traumatic dementia, with preference for dementia of Alzheimer type.

Essential oils from rosemary, red thyme, bergamot, pine needle, lavender, and cypress, used as the active ingredients of the present invention, have found applications in aromatherapy and cosmetics. Since the essential oils may be prepared using typical extraction and separation methods, or are commercially available, a description of their extraction from plants is omitted in the present invention.

Cypress essential oils have temporal analgesic effects on muscle cramps, myalgia, and arthritis, and are helpful in immunopotentiation, and treatment of strain, stress, lethargy, and neurasthenia. In addition, an oil is used as an ingredient of after-shave skin lotion thanks to its sterile, stringent, and refreshing effects.

Rosemary essential oils exhibit invigorant, digestive, stringent, antiflatulent, and antibacterial activities, and are known for their headache relief, cerebral circulation, memory enhancement and attention improvement. In addition, it is applied to a stringent skin lotion thanks to an anti-aging effect, and to a hair care shampoo or rinse thanks to anti-dandruff treatment.

Red thyme essential oils are used where cardiotonic, invigorant, expectorant, anthelmintic, antiflatulent, antihypertensive, anti-toxic, anti-rheumatoidal, aphrodisiac, germicidal, insecticidal, disinfectant, appetizing, anti-dysmenorrhea, or diuretic effects are needed. Also, a red thyme essential oil is known for activating brain cells and improving memory and attention.

Containing an antibacterial ingredient of the leaf, bergamot essential oils exhibit anti-inflammatory and anti-bacterial activity. Also, a bergamot essential oil gives a sensation of refreshment and functions to control sebum, which allows it to be widely applied to functional cosmetics for the treatment of dermal diseases such as acne. Further, the oil is used to retard and prevent skin aging because of its excellent skin soothing and antioxidant effects.

Pine needle essential oils are known as exhibiting expectorant, stomachic, perspirative, sterilizing, disinfectant, anti-inflammatory, and deodorant activities, and are useful in the treatment of dermatitis, such as stasis dermatitis, eczema, psoriasis, and the like.

Lavender essential oils, obtainable from various kinds of lavender including English lavender, French lavender, and spike lavender, have long been used for aromatherapy. There are a variety of uses of lavender essential oils, including sterilization, disinfection, preservation, moss proofness, hypnosis, sedation, and pain relief, and thus they are used for the treatment of headache, insomnia, depression, anxiety, impatience, bronchitis, neuralgia, rheumatism, myalgia, burn, and insect bites. Particularly, a skin lotion comprising a lavender essential oil is known to soothe the skin, and exert moisturizing, regenerative, and rinsing effects on the skin.

For use in comparison with the natural essential oils of the present invention, synthetic perfumes (artificial perfumes) were prepared from combinations of fragrance of various images, including floral aromatics reminiscent of flowers, green aromatics associated with clean leaves and nature, and fruity aromatics for sweet, and fresh sensations.

In accordance with another aspect thereof, the present invention addresses an aromatic cosmetic composition for the enhancement of memory and the prevention and improvement of cognitive dysfunction, comprising a cypress essential oil complex.

Preferably, the first essential oil component and the second essential oil component are used together in an amount of from 0.01% by weight to 100% by weight based on the total weight of the aromatic cosmetic composition. In another preferable embodiment, the second essential oil component is used in an amount of from 0.01 part by weight to 100 parts by weight based on 100 parts by weight of the first essential oil component. However, because the cosmetic composition of the present invention takes advantage of fragrance in the enhancement of memory and the prevention and improvement of cognitive dysfunction, composition ratios of ingredients are not as significant as those in oral formulations. The composition ratio may vary depending on the patient's state, and the kind and severity of the disease to be treated.

In one embodiment of the present invention, the aromatic cosmetic composition may further comprise a suitable carrier, excipient, or diluent. Examples of the carrier, excipient, and diluent useful in the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil, but are not limited thereto.

The aromatic cosmetic composition of the present invention may be formulated into various forms, including externals, such as a shampoo, rinse, perfume, lotion, cream, skin lotion, aromatic, aerosol, and the like, and as a detergent, mousse, hair spray, deodorant, or ointment. For the formulation, a diluent or excipient such as a filler, a thickener, a binder, a wetting agent, a disintegrant, a surfactant, and the like, may be employed. In addition to an excipient, a lubricant such as magnesium stearate, talc, etc. may also be used.

A better understanding of the present invention may be obtained through the following examples which are set forth to illustrate, but are not to be construed as limiting the present invention.

Experimental animals were c57/BL6 male mice (8 weeks old, Hyochang Science, Daegu, Korea), each weighing 20-25 g, that had been acclimated to experiment environments before experiment. The mice were divided into 15 groups of five, as shown in Table 1, below. They were exposed to fragrance for three hours per day during the acclimation and experiment periods. The experimental animals were raised at a temperature of 21˜26° C. and a relative humidity of 40˜60%, with a light/dark cycle of 12/12 hrs, in an animal breeding room of the Daegu Haany University.

In the cypress essential oil complexes (I, J, K, L, and M) used in the following experiments, a cypress essential oil was mixed at a weight ratio of 1:1 with another essential oil.

Essential oils of rosemary, red thyme, bergamot, pine needle, lavender, and cypress were provided from Aroma Bank, while artificial floral, fruity, and green aromatics were purchased from Sung Woo International. The experimental animals of Table 1 were placed in specially designed fragrance cages which were previously scented with the fragrance of the essential oils for 30 min.

TABLE 1

| Group | Status |
|---|---|
| A | Sham group neither administered with scopolamine, nor |
| | exposed to fragrance |
| B | Control administered with scopolamine |
| C | Exposed to the fragrance of rosemary essential oil for |
| | 3 hrs before administration with scopolamine |
| D | Exposed to the fragrance of red thyme essential oil |
| | for 3 hrs before administration with scopolamine |
| E | Exposed to the fragrance of bergamot essential oil for |
| | 3 hrs before administration with scopolamine |
| F | Exposed to the fragrance of pine needle essential oil |
| | for 3 hrs before administration with scopolamine |
| G | Exposed to the fragrance of lavender essential oil for |
| | 3 hrs before administration with scopolamine |
| H | Exposed to the fragrance of cypress essential oil for |
| | 3 hrs before administration with scopolamine |
| I | Exposed to the fragrance of cypress essential oil + |
| | rosemary essential oil for 3 hrs before administration |
| | with scopolamine |
| J | Exposed to the fragrance of cypress essential oil + |
| | red thyme essential oil for 3 hrs before |
| | administration with scopolamine |
| K | Exposed to the fragrance of cypress essential oil + |
| | bergamot essential oil for 3 hrs before administration |
| | with scopolamine |
| L | Exposed to the fragrance of cypress essential oil + |
| | pine needle essential oil for 3 hrs before |
| | administration with scopolamine |
| M | cypress essential oil + lavender essential oil for 3 hrs |
| | before administration with scopolamine |
| N | Exposed to the fragrance of synthetic floral + fruity |
| | aromatics for 3 hrs before administration with |
| | scopolamine |
| O | Exposed to the fragrance of synthetic floral + fruity + |
| | green aromatics for 3 hrs before administration with |
| | scopolamine |

A systemic examination was made of the influence of cypress essential oil on the enhancement of memory and the treatment and prevention of cognitive dysfunction. In this context, behavioral tests were performed on experimental animals in which memory impairment was induced by scopolamine, so as to analyze their memory. Scopolamine is known as an acetylcholine receptor antagonist that is suppressive of memory. Experimental animals were exposed for 3 hrs per day to essential oil fragrance from 5 days before exposure to scopolamine (S0929, Sigma-Aldrich Co., U.S.A.). Then, scopolamine was intraperitoneally injected at a dose of 1 mg/kg to induce memory impairment before performing a Y-maze test, a Morris water-maze test, a passive avoidance test, and a fear conditioning test.

### EXPERIMENTAL EXAMPLE 1

### Y-Maze Test

To examine the effect of the essential oils of the present invention, a modified Y-maze test was conducted as follows.

Testing was carried out in a Y-shaped maze with three arms (6 cm in width, 28 cm in length, 18 cm in height) at a 120° angle from each other. After introduction to the end of one arm, the mouse was allowed to freely explore the three arms. The number and order of arm entries were recorded in order to calculate the percentage of spontaneous alternations. It was determined by the number of triads, that is, the number of cases where the mouse entered the three arms sequentially, e.g., in the order of ABC, BCA, CAB, etc., in one trial, as calculated by the following Math Formula 1:

$$\% of Spontaneous Alteration = \frac{No of Triads}{Total No . of Arm Entries - 2} \times 100 \qquad [ Math Formula 1 ]$$

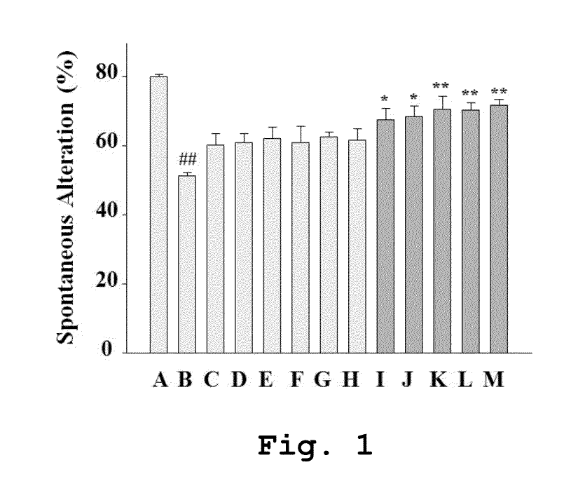

The results of the Y-maze test are depicted in FIG. 1 and summarized in Table 2. As is understood from the data of FIG. 1 and Table 2, statistical significance was observed between the sham group (A) and the control administered with scopolamine alone (B). That is, the mice intraperitoneally injected with scopolamine alone (B) exhibited an average spontaneous alteration of 51.30±1.0% as a result of the induction of memory impairment, whereas the sham group (A) recorded an average percentage of spontaneous alteration at 79.90±0.69. On the other hand, the percentage of spontaneous alteration of the test groups C to H, which were exposed respectively to rosemary, red thyme, bergamot, pine needle, lavender, cypress essential oils, were read in the sixties, thus showing that the mice exposed to the fragrance of the essential oils were improved in cognitive ability, compared to the control administered with scopolamine alone (B). In addition, higher measurements were obtained when the mice were exposed to the fragrance of the compositions of the presence invention comprising a cypress essential oil in combination with an essential oil selected from among a rosemary essential oil, a red thyme essential oil, a bergamot essential oil, a pine needle essential oil, and a lavender essential oil (I to M), compared to individual essential oils. Particularly, when inhaling the fragrance of a combination of cypress essential oil plus bergamot, pine needle, or lavender essential oils (K, L, M), the mice were found to be further more improved in memory.

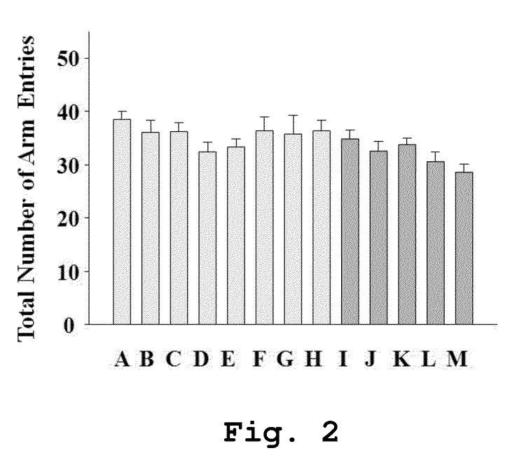

As for the total number of arm entries, it was not significantly different among the groups under the same experiment conditions, as shown in FIG. 2.

TABLE 2

| Group | % of Spontaneous Alteration |
|---|---|
| A | 79.90 ± 0.69 |
| B | 51.30 ± 1.0 |
| C | 60.29 ± 3.12 |
| D | 60.80 ± 2.80 |
| E | 62.13 ± 3.24 |
| F | 62.52 ± 1.53 |
| G | 60.93 ± 4.65 |
| H | 61.67 ± 3.27 |
| I | 67.48 ± 3.29 |
| J | 68.45 ± 3.06 |
| K | 70.45 ± 3.95 |
| L | 70.29 ± 2.22 |
| M | 71.63 ± 1.83 |

Although a combination of cypress essential oil plus two or more selected from among rosemary, red thyme, bergamot, pine needle, and lavender essential oils was not employed in this experiment, it is expected to allow for at least the same effect, compared to the use of individual single oils, from the results obtained above.

### EXPERIMENTAL EXAMPLE 2

### Morris Water-Maze Test

In a Morris water-maze test, the essential oils of the present invention were examined for influence on spatial learning and short and long-term memory recovery. The maze set up contained a round water pool (stainless steel, 45 cm deep with a diameter of 120 cm), and a hidden rescue platform (30 cm high with diameter of 10 cm). The pool was filled with water (22±2° C.) to a height 2 cm higher than the platform, so that the mouse was rescued when it had a seat on the platform. Because the water-maze test is designed to examine the ability of the subject to search for the platform depending on the memory of surrounding environments, no changes were made in the surrounding environments during the experiment period. When the subject stayed for 20 sec or longer on the platform, the time to reach the platform was recorded as escape latency. Escape latency values of the measurements obtained in daily three test rounds for four days were averaged (mean escape latency). The mice were monitored under a camera installed above the water pool, with the escape latency recorded with a computer program (Ethovision 3.1, Noduls, Netherland). Each animal underwent three consecutive trials per day for four days. Starting directions were set to differ from one trial to another so as to minimize the likelihood that the subject might find the platform by chance. If the animal did not reach the platform within 120 sec, the escape latency for this trial was recorded as 120 sec. When the animal reached the platform, it was allowed to sit on the platform for 20 sec in order that it recognized and remembered environmental clues.

A far lower mean escape latency was observed in the test groups C to M, exposed to the essential oils, than the control (B) administered with scopolamine alone, indicating that the essential oils significantly improve spatial learning and cognitive ability. Particularly, test groups I to M, exposed to cypress essential oil complexes according to preferable embodiments of the present invention, had memory that was recovered to a greater extent than did the test groups C to H, exposed to single essential oils.

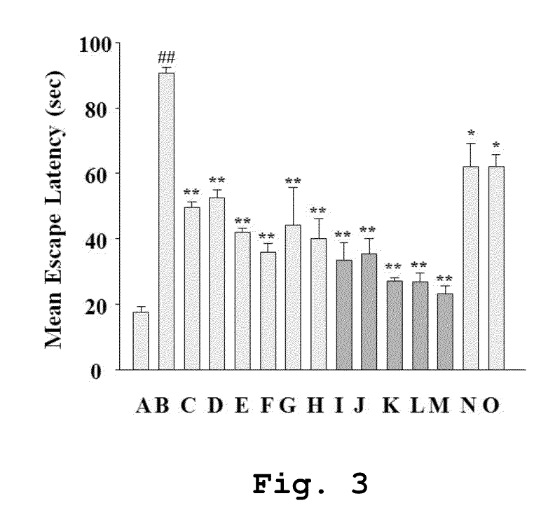

When fragrance of a combination of synthetic floral and fruity aromatics (N), and a combination of floral, fruity, and green aromatics (O) was used, mean escape latency was recorded as 61.93±7.27 sec, and 62.2±3.64 sec, respectively, indicating that synthetic fragrance has poorer effects than does the natural fragrance (see FIG. 3 and Table 3).

TABLE 3

| Group | Mean Escape Latency (Unit: sec) |
|---|---|
| A | 17.67 ± 1.58 |
| B | 90.63 ± 1.72 |
| C | 49.67 ± 1.69 |
| D | 52.6 ± 2.33 |
| E | 42.13 ± 1.18 |
| F | 35.87 ± 2.71 |
| G | 44.33 ± 11.35 |
| H | 40.0 ± 6.22 |
| I | 33.6 ± 5.40 |
| J | 35.4 ± 4.59 |
| K | 27.13 ± 0.98 |
| L | 26.93 ± 2.72 |
| M | 23.33 ± 2.31 |
| N | 61.93 ± 7.27 |
| O | 62.2 ± 3.64 |

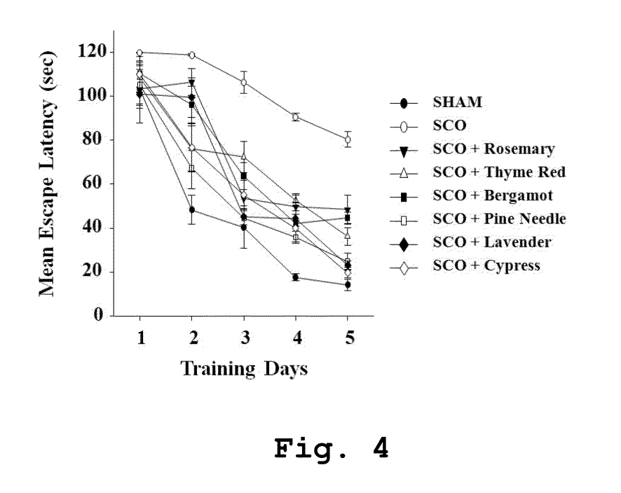

Particularly, the sham group and the test groups were decreased in mean escape latency, with stability, after Day 3 of training, indicating the recovery of long-term memory during the period of experiments (see FIG. 4). Moreover, exposure to a combination of cypress essential oil plus one of bergamot, pine needle, and lavender essential oils (K, L, M) brought about higher improvement in memory.

### EXPERIMENTAL EXAMPLE 3

### Passive Avoidance Test

A passive avoidance test was performed in a training chamber which was divided into two compartments (each 25 cm in width and 20 cm in length with a height of 20 cm) separated by a guillotine door: one compartment was lighted by an overhead light while the other remained dark. In the dark compartment, stainless rods, 2 mm thick, were installed at regular intervals of 1 cm on the bottom so as to deliver an electrical footshock. A mouse was placed in the lighted compartment, facing away from the dark compartment and allowed to explore for 10 sec. After 10 sec, the guillotine door was opened for 20 sec and the mouse was allowed to explore freely. When the mouse entered the dark compartment with all four paws, the guillotine door was closed, and a footshock (0.5 mA, 5 sec duration) was delivered (acquisition trial). On test day (24 hours after training), the mouse was returned to the lighted compartment, facing away from the dark compartment, and was allowed to meet the same situation. The latency to enter the dark compartment (step-through latency time) was recorded (from the time the door was lifted) to a maximum of 300 sec (retention trial).

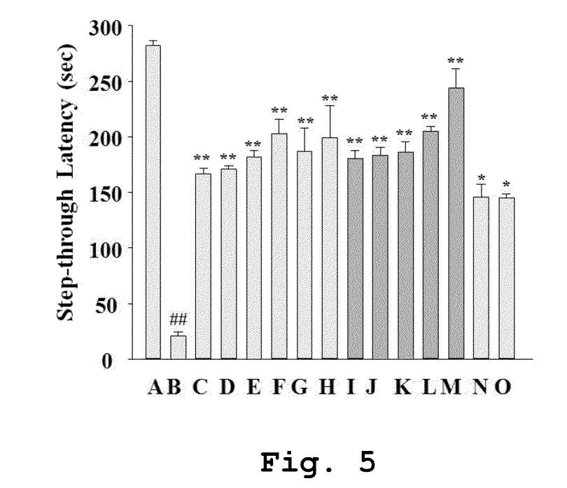

The test results are depicted in FIG. 5 and summarized in Table 4. During the retention trial on day 2, the step-through latency time was recorded as 281.86±4.46 sec for the Sham group (A) and 21.15±3.32 sec for the control (B), with statistical significance therebetween. From these data, it was fully understood that memory impairment was certainly induced in the control. Test groups C to H which were allowed to inhale the fragrance from respective rosemary, red thyme, bergamot, pine needle, lavender, and cypress essential oils, and test groups I to M which had been exposed to the fragrance from respective combinations of cypress essential oil plus one of rosemary, red thyme, bergamot, pine needle, and lavender essential oils were found to be improved in memory, with a peak memory detected in the test group M treated with cypress essential oil+lavender essential oil.

When the mice were injected with scopolamine after exposure for 3 hrs to fragrance of a combination of synthetic floral and fruity aromatics (N), and a combination of floral, fruity, and green aromatics (O), the step-through latency time was measured at 146.06±10.91 sec, and 145±3.79 sec, respectively, indicating that synthetic fragrance has poorer effects than does the natural fragrance.

TABLE 4

| Group | Step-through Latency Time (Unit: sec) |
|---|---|
| A | 281.86 ± 4.46 |
| B | 21.15 ± 3.32 |
| C | 166.6 ± 5.15 |
| D | 171.12 ± 2.79 |
| E | 181.86 ± 5.90 |
| F | 202.64 ± 12.88 |
| G | 187.04 ± 20.78 |
| H | 199.36 ± 28.27 |
| I | 183.29 ± 7.48 |
| J | 186.38 ± 9.20 |
| K | 186.38 ± 9.20 |
| L | 205.04 ± 4.19 |
| M | 243.52 ± 17.82 |
| N | 146.06 ± 10.91 |
| O | 145.00 ± 3.79 |

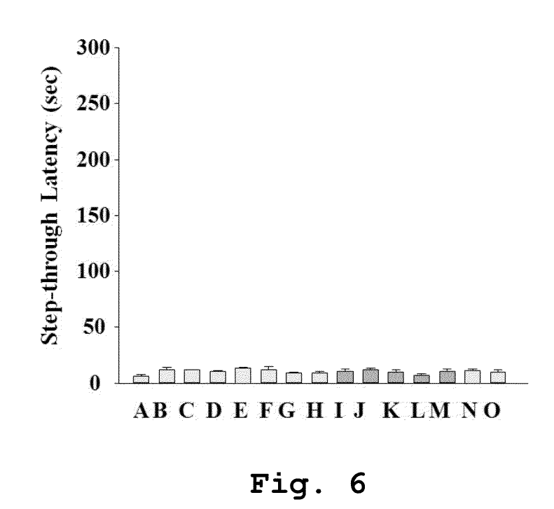

During acquisition trial on day 1, no significant differences in step-through latency time were observed among groups (see FIG. 6).

### EXPERIMENTAL EXAMPLE 4

### Fear Conditioning Test

A fear conditioning test was performed to examine the learning and memory of the animal model in which memory impairment had been induced by scopolamine, in a classical conditioning paradigm in which an environmental contextural or conditional stimulus (CS) was associated with an aversive unconditional stimulus (US, electric shock). Fear conditioning was carried out by either contextual fear conditioning or cued fear conditioning.

The mice were trained twice at regular intervals of 150 sec with pairings of sound stimulus (CS, 15 sec, 68-80 dB, 30 kHz) and electric shock (US, 1 sec, 0.5 mA). Sixty seconds after the final electric shock (US), the mice were withdrawn from the conditional chamber. After 24 hours, the following tests were carried out. Contextual conditioning: When mice were placed in the conditional chamber without CS presentation, time spent freezing was recorded and served as a baseline for conditioned fear response to the context (hippocampus-dependent memory). As used herein, the term “freezing” is defined as a condition under which an animal does not carry out any behavior except for breathing. Cued conditioning: two hours later, the context was changed. The mice were placed in a new different chamber, and presented with the same sound stimulus as that of the training at which time spent freezing was measured, and used as a baseline for conditioned fear response to cue (amygdala-dependent memory).

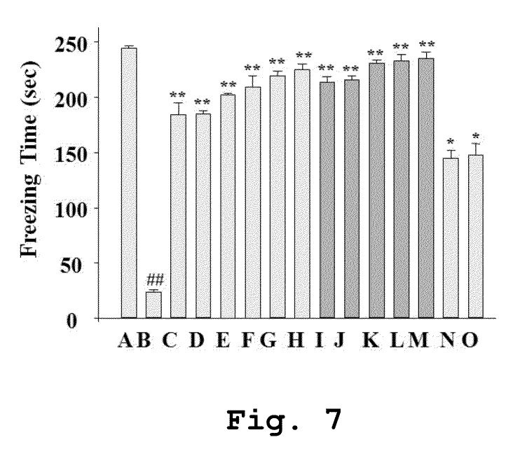

Fear conditioning test results are given in FIG. 7 and Table 5. As can be seen, there were statistical significances among the sham group (A), the control administered with scopolamine alone (B), and the test groups C to M administered with scopolamine after exposure to the essential oils. In the contextual fear conditioning, the time of freezing behavior was recorded as 244.5±2.42 sec for the sham group (A), but as 23.77±2.30 sec for the control (B) because memory impairment was induced by intraperitoneal injection of scopolamine (FIG. 7). On the other hand, the test groups which inhaled the essential oils were improved in cognitive ability, with statistical significance, compared to the control, as shown in Table 5, below. On the whole, the compositions of the present invention (I to M) exhibited better effects than did the compositions comprising single essential oils (C to H).

TABLE 5

| Group | Time of Freezing Behavior (Unit: sec) |
|---|---|
| A | 244.5 ± 2.42 |
| B | 23.77 ± 2.30 |
| C | 184.6 ± 10.52 |
| D | 185.4 ± 2.71 |
| E | 201.8 ± 1.85 |
| F | 209.4 ± 10.10 |
| G | 219.2 ± 4.90 |
| H | 225.0 ± 5.30 |
| I | 213.5 ± 5.24 |
| J | 215.7 ± 3.57 |
| K | 230.6 ± 3.23 |
| L | 233.0 ± 5.66 |
| M | 235.4 ± 5.61 |
| N | 145.0 ± 6.99 |
| O | 147.8 ± 10.11 |

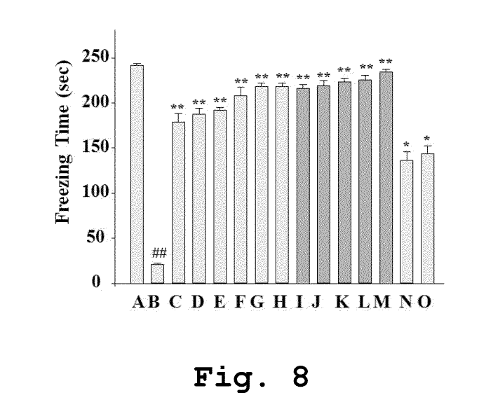

In the cued fear conditioning, the time of freezing behavior was measured as 21.23±1.42 sec on average for the control (B), intraperitoneally injected with scopolamine alone, and as 241.42±2.02 sec on average for the sham group (FIG. 8 and Table 6). The test groups which were allowed to inhale the essential oils were found to have increased cognitive ability, with statistical significance, compared to the control. Particularly, exposure to a combination of cypress essential oil plus one of bergamot, pine needle, and lavender essential oil (K, L, M) afforded higher improvements in memory. The fragrance of a combination of synthetic floral and fruity aromatics (N), or a combination of floral, fruity, and green aromatics (O) was apt to bring about lower effects, compared to the natural fragrance.

TABLE 6

| Group | Time of Freezing Behavior (unit: sec) |
|---|---|
| A | 241.42 ± 2.02 |
| B | 21.23 ± 1.42 |
| C | 178.6 ± 9.87 |
| D | 187.8 ± 6.12 |
| E | 191.6 ± 3.12 |
| F | 207.8 ± 9.40 |
| G | 218.0 ± 3.94 |
| H | 226.3 ± 7.14 |
| I | 216.3 ± 4.23 |
| J | 219.2 ± 5.57 |
| K | 223.4 ± 3.89 |
| L | 225.2 ± 5.53 |
| M | 234.2 ± 3.06 |
| N | 136.6 ± 9.37 |
| O | 144.0 ± 8.44 |

1. A cosmetic composition for enhancement of memory and improvement of cognitive dysfunction, comprising a primary essential oil component composed of a cypress essential oil, and a secondary essential oil component selected from the group consisting of a pine needle essential oil, a lavender essential oil, a rosemary essential oil, a red thyme essential oil, a bergamot essential oil, and a combination thereof.

2. The cosmetic composition of claim 1, wherein the primary essential oil component and the secondary oil component are used together in an amount of from 0.01% by weight to 100% by weight, based on a total weight of the composition.

3. The cosmetic composition of claim 1, wherein the secondary essential oil component is used in an amount of from 0.01 part by weight to 100 parts by weight, based on 100 parts by weight of the primary essential oil component.

4. The cosmetic composition of claim 1, further comprising an additive selected from the group consisting of a carrier, an excipient, a diluent, a filler, a thickener, a binder, a wetting agent, a lubricant, a disintegrant, a surfactant, and a combination thereof.

5. The cosmetic composition of claim 1, being in a form of a formulation selected from the group consisting of a shampoo, a rinse, a perfume, a lotion, a cream, a skin lotion, a detergent, a mousse, a spray, a hair wax, a fragrant, a deodorant, and a hair gel.
